(19) United States
(12) Patent Application Publication
(10) Pub. No.: US 2005/0227222 A1
(43) Pub. Date: Oct. 13, 2005

(54) Pathogen identification method

(75) Inventors: Jerome J. Braun, Boxborough, MA (US); Yan Glina, Revere, MA (US); Nicholas Judson, Somerville, MA (US); Rachel Herzig-Marx, Newtonville, MA (US)

Correspondence Address: FISH & NEAVE IP GROUP;ROPES & GRAY LLP, ONE INTERNATIONAL PLACE, BOSTON, MA, 02110-2624, US

(73) Assignee: Massachusetts Institute of Technology, Cambridge, MA (US)

(21) Appl. No.: 10/822,562

(22) Filed: Apr. 9, 2004

(51) Int. Cl. C12Q 1/00; C12Q 1/70; C12Q 1/68; G01N 33/53; G01N 33/567; G01N 33/554; G01N 33/569

(52) U.S. Cl. 435/4; 435/5; 435/7.32; 702/19; 435/6

(57) ABSTRACT

Methods and apparatuses for detecting, identifying, characterizing, and tracking pathogenic agents in any of a wide range of samples.

### GOVERNMENT SUPPORT

This invention was supported, in whole or in part, by Lincoln Contract Number F19628-00-C-0002 from The United States Air Force. The Government has certain rights in the invention.

### BACKGROUND

Today, scientists and engineers have developed technologies that allow biological data to be collected on a large scale. The most common example of this kind of technology is the microarray. Microarrays allow a biological sample to be tested for the presence of hundreds to thousands of biological compounds. (See e.g., Marton et al., 1998, Drug Target Validation and Identification of Secondary Drug Target Effects Using Microarrays, Nature Medicine in Press).

Microarrays provide the ability to generate a profile of the different cellular constituents present in a cell or other biological entity. In some cases, a microarray can measure 100,000 or more characteristics of a cell or entity. Given the large number of characteristics, the profile of a particular cell or biological entity is typically quite complex.

To understand how a cell is working or responding to a change in environment or a new condition, scientists need to understand how these profiles change. Given the number of features of a cell or entity that can be measured and analyzed, scientists are often overwhelmed by the volume of data they need to process to recognize a response of interest. To this end, the scientists typically try to reduce the feature space and therefore the number of characteristics—that they are analyzing to reduce the complexity of the problem. To this end, scientists and engineers have developed various feature extraction and selection technologies that allow scientists to analyze the profile data and to identify a reduced set or portion of measured responses that need to be analyzed for the scientist to feel that they have identified the biologically significant change in the profile. One example of this is a feature selection process that tries to determine from an understanding of the biological response of the cell structure, which genes are most likely involved in a particular reaction or response. These biologically relevant genes are measured, along with many others, but the scientists concentrate their understanding and efforts on the changes that occur to this limited set of genes. In other techniques, scientists apply a principal component analysis to the measured data to identify mathematically a reduced set of genes that seem to characterize the most significant or significant response of the cell. In either case, the effort is directed to reducing the number of genes, proteins, or other characteristics of the cell or entity that are to be analyzed. In this way, the scientists hope to identify a reduced set of characteristics or responses that can be more easily analyzed yet still provide biologically relevant information.

Although these feature selection and identification processes can be quite effective, they still require that the scientist or engineer have an underlying understanding of the biological action being studied. Accordingly, there is a need in the art for systems and methods capable of profiling and recognizing biological activity or entities wherein the underlying biological activity is unknown or undetermined.

### SUMMARY

The invention, among other things, provides methods and apparatus which can be used to identify, classify, and/or track pathogenic agents that may infect or otherwise contaminate animals, humans, water sources, air sources, or soil sources. Pathogenic agents that can be identified, classified, and/or tracked using the methods and apparatuses described herein, include but are not limited to, nucleic acid containing pathogenic agents such as bacteria, viruses, fungi, and protozoa. However, the pathogenic agents that can be identified, classified, and/or tracked using the described methods and apparatuses also include non-nucleic-acid-based agents such as toxins. The systems and methods have a wide range of forensic, medical, epidemiological, environmental, industrial, public health, and anti-bioterrorism applications.

The systems and methods described herein will employ, unless otherwise indicated, conventional techniques of cell biology, cell culture, molecular biology, transgenic biology, microbiology recombinant DNA, and immunology, which are within the skill of the art. Such techniques are described in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular: Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y:, 1986).

Other features and advantages of the invention will be apparent from the following detailed description, and from the claims.

### DETAILED DESCRIPTION OF THE DRAWINGS

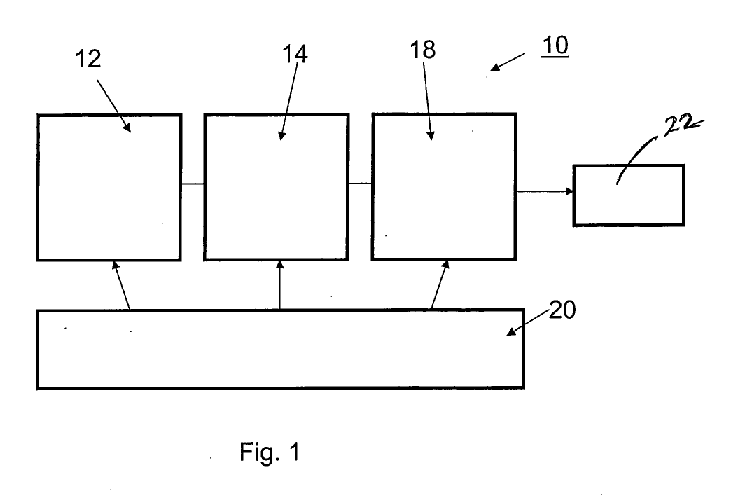

FIG. 1. Depicts a high-level functional block diagram of one system according to the invention;

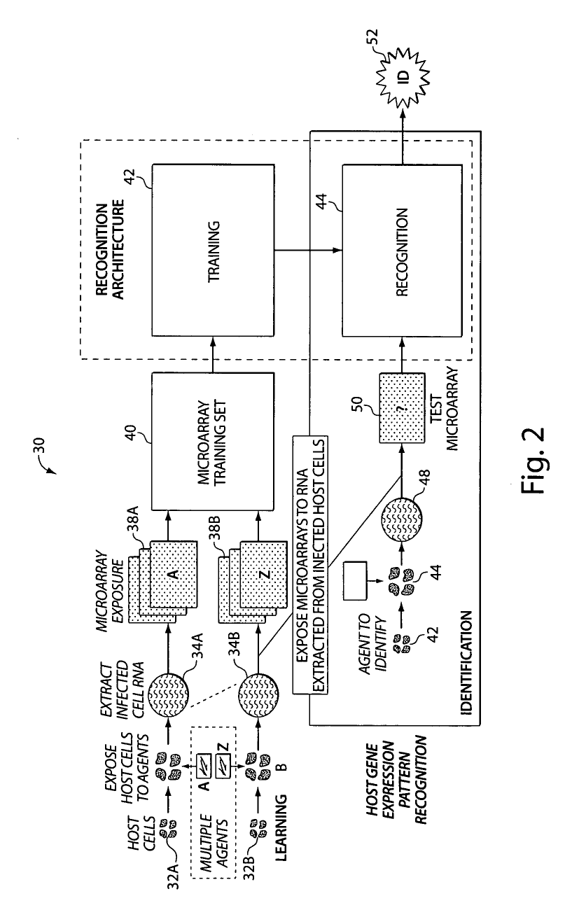

FIG. 2. Depicts in more detail one exemplary embodiment of a system according to the invention;

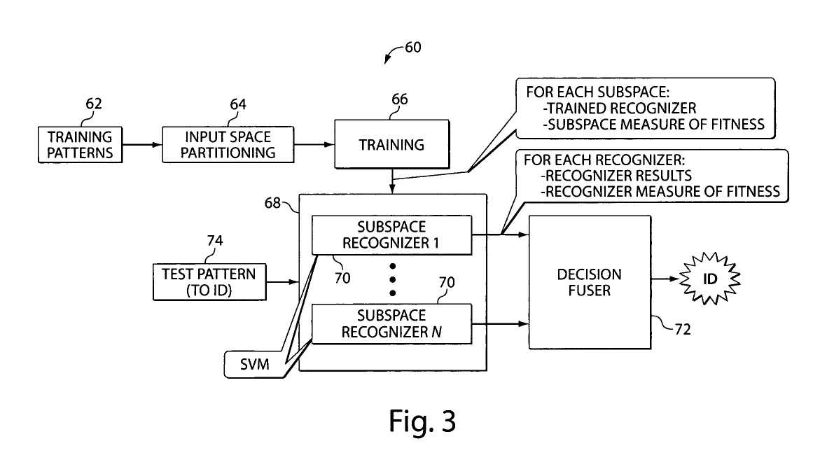

FIG. 3. Depicts one particular embodiment of a system according to the invention;

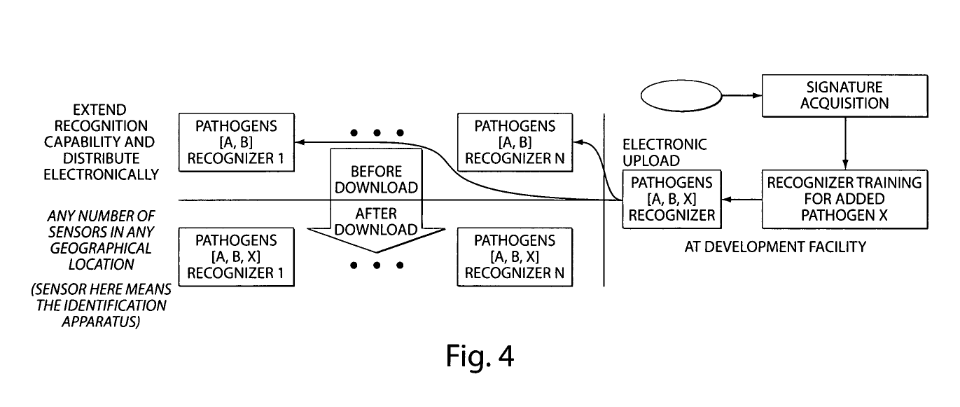

FIG. 4. Illustrates a data flow diagram of adding a pathogen signature to a system according to the invention to extend the recognition capability of such a system.

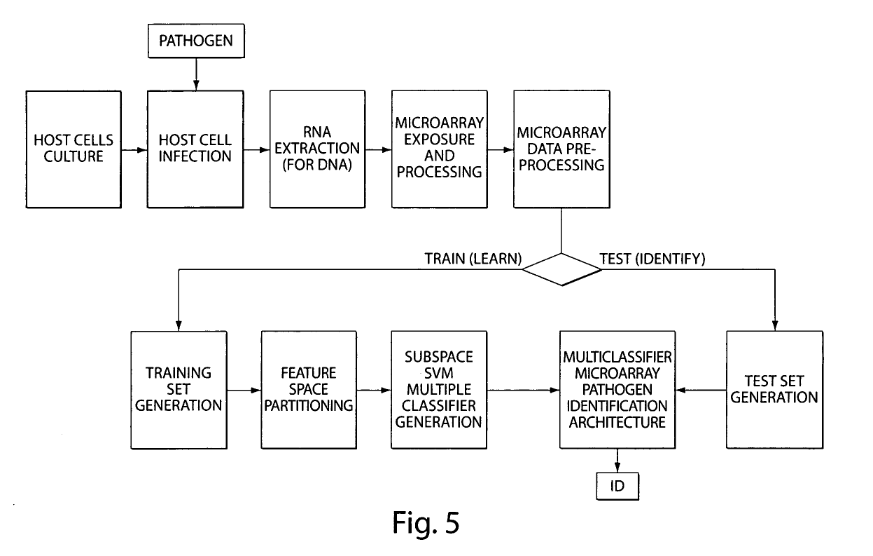

FIG. 5. Depicts a data flow diagram of one process according to the invention for training and employing a recognizer according to the invention.

### DETAILED DESCRIPTION

The systems and methods described herein provide, among other things, systems and methods for automatic identification of multiple pathogens, including unsequenced and unknown or uncataloged species. It is suitable for a wide range of pathogens including nucleic acid containing pathogens such as bacteria and viruses, and also pathogenic materials that do not possess their own genomic structures, e.g., toxins. The present methods have a range of applications including, but not limited to, defense and civilian applications. In biological defense, the invention can be used for detection and identification of biological agents used, for example, during an attack. Civilian applications include medical diagnostics in clinical laboratories, hospitals, or physicians offices. Additionally, because of its ability to operate on unknown or uncataloged species, its applications include health surveillance, such as tracking a spread of an outbreak caused by such an agent.

The invention, in one embodiment, provides pathogen identification via information fusion and automatic recognition of microarray patterns representing an in vitro gene expression of cultured host cells responding to a pathogen. The systems and methods combine pattern recognition, information fusion, the use of cultured host cells and their in vitro expression patterns, and microarray-based sensing. The combined effect of these components [i.e., 1 use of cultured cells as hosts and their in vitro expression pattern, 2: microarray-based sensing 3: pattern recognition methods, and 4: information fusion] in the specific application context create an improved process for automatic and versatile identification of pathogenic agents. In particular, the invention provides methods for identifying, in a uniform manner, a variety of pathogenic material types (including bacterial and viral as well as material without genomic structures, such as toxins, etc.). Finally, it constitutes a method for identifying unsequenced pathogens, as well as a method for discovering and/or tracking occurrences of an unknown or uncataloged pathogen, such as during outbreaks of new or previously undocumented disease.

FIG. 1 depicts as a block diagram a high level view of one system according to the invention that includes cultured cell hosts, microarray based sensing, pattern recognition and information fusion. Specifically, FIG. 1 depicts a system 10 that includes a first functional block 12 representative of a set of cultured cells employed as hosts and capable of providing in vitro expression patterns. Block 14 is representative of a microarray device capable of sensing the response of the host cells 12. A pattern recognition process 18 communicates with the microarray and an information fusion process 20 can interact with one or more of the above described elements for the purpose of providing a robust identification 22 of the pathogenic agent to which the host cells were exposed. Thus, the system 10 is a functional block diagram that depicts a pathogen identification system that combines the use of host cells and in vitro expression patterns, microarray sensing, machine learning and recognition, and information fusion to capture and process all or substantially all of the expression information generated by the host cells and employ that information to create a robust identification of the pathogen that has affected the biological response of those cells.

The host cells 12 serve as an amplification mechanism since a small quantity of a virulent pathogen may result in a vigorous expression response in the host cells. They also serve as a filter of differences in the pathogens genome, such as those caused by some mutations or genetic engineering, since the host expression response pattern is likely to be similar if these differences do not significantly alter the pathogen-host interaction. Use of cultured cells results in an abundant source of readily available, reasonably reproducible host cells. Furthermore, the use of cultured cells offers an additional filter because variations in responsiveness to pathogenic agents that one would expect to observe across various infected organisms is greatly diminished. In one embodiment, the host cells 12 are human alveolar epithelial cells A549. However, the systems and methods described herein can employ other types of cells.

The microarrays employed may be any suitable microarrays and will be discussed in more detail hereinafter. The microarrays can be chips that provide a solid substrate with a plurality of micro structures or micro-scale structures on which certain processes, such as physical, chemical, biological, biophysical or biochemical processes, etc., can be carried out know in the art and literature including the Moreover, although FIG. 1 depicts a system 10 that employs microarrays to collect data from the host cells, it will be understood that the invention is not so limited and that other suitable systems and techniques for collecting response data may be employed without departing from the scope of the invention.

The approach depicted in FIG. 1 takes advantage of all or substantially all information contained in the microarray patterns, produced by microarrays 14 rather than relying on the mostly questionable and sometimes entirely unavailable knowledge of gene “relevance”. The machine learning process 18 implies that pathogen signatures are learned automatically from examples that consist of microarray patterns obtained by exposure of microarrays 14 to the labeled cDNA resulting from the RNA of host cells 12 responding to the pathogen. RNA extraction and conversion to cDNA is the approach employed during experimental work to date; however, other molecular phenomena/products of host cell infection may be used if different microarray modalities (e.g., proteomic) are utilized, a possibility enabled by the information fusion aspects of the approach. In addition, it is understood that in certain other practices, the pathogens themselves may be processed and the microarray patterns generated directly from the pathogen expression.

The techniques related to the information fusion process include the use of multiple classifiers and the technique referred to in this approach as the input space partitioning. These, together with the pattern recognition techniques (for example, Support Vector Machines) are intended to accommodate the small sample size scenarios that arise in this pathogen-identification setting. While the method does not exclude limiting the number of inputs (spots), one of the facets stemming from the information fusion process 20 is the exploitation of all or substantially all available information. This contrasts with approaches that attempt to determine a-priori the genes whose expression can be utilized for the recognition process. Rather than narrowing the input space, the approach described herein seeks to widen it, aiming to exploit subtle signature patterns in the large and diverse input (spot) space. This approach also contrasts with the feature selection type approaches, in which many of the inputs are discarded and only those that pass some type of relevance tests are used in the recognition process. This approach allows avoiding the need for input discarding, that is unreliable and can lead to discarding important information especially in the small sample size situations. The exploitation of all or substantially all information sources approach is beneficial in situations where the knowledge underlying molecular biological phenomenology is unavailable, as is the case for many pathogens. This broadens the expected applicability of the approach. Moreover, the information fusion process 20 of the approach facilitates the following embodiments of the invention: (a) use of multiple types of host cells derived from different tissue, cell, or organ sources (e.g., lung, liver, skin, neuronal, etc.); (b) use of cells derived from multiple organisms (e.g., human, non-human primate, mouse, rat, plant, yeast, etc.); (c) side-by-side use of multiple microarrays of different probe content; and (d) use of expression modalities other than genomic (e.g., genomic microarrays, proteomic microarrays, both genomic and proteomic microarrays, etc.) All of these may participate in any combination within the fusion framework. In other words, the fusion approach allows simultaneous use of, e.g., multiple microarrays of different structures, different types, such as genomic and proteomic, and one or more cell types.

While the current experiments involve one cell type (human alveolar epithelial cells A549) and a few exemplary pathogens (P. aeruginosa and Influenza A virus), the invention contemplates the use of all of the above, including any of a number of host cell types, and the identification of any of a number of pathogenic agents.

Turning to FIG. 2, there is depicted a system 30 that represents one particular embodiment over the systems and methods described herein. As shown, the system 30 includes a set of host cells, 32A and 32B. The host cells can be exposed to pathogenic agent (e.g., a sample containing a pathogenic agent) to infector otherwise affect the cells and the infected (affected) cell RNA 34A can be extracted, converted to cDNA and hybridized to the microarrays 38A. In the depicted embodiment, there are a plurality of microarrays 38A. However, in other embodiments, there may only be a single microarray. The extent of this plurality varies between different embodiments. Also shown in FIG. 2 is that a parallel tract exists. Specifically, another set of host cells, 32B, of the same type of host cells as 32A in one embodiment, can also be exposed to a set of agents and the infected cell RNA in 32B can be removed and the resulting cDNA applied to the microarrays 38B. FIG. 2 depicts two parallel tracts. However, it will be apparent to those of ordinary skill in the art that the number of tracts employed can vary according to the invention and the depicted parallel tracts are merely figurative in that host cell exposure and RNA extraction and application to a microarray can occur for different pathogenic agents sequentially. After each sequence of exposure, infection, and microarray exposure, the exposure data collected can be applied to form a microarray training set 40.

The microarray training set 40 can comprise a set of electronic data files that represent the patterns identified on the microarrays for a particular pathogenic agent. In one embodiment, the microarray training set is a computer data file that may be either a flat file or a data base. In either case, the microarray training set includes a set of pathogen signatures that can be used as a training set in the training process 42.

The training process 42 may be a computer process of the type commonly employed for training a recognition unit or a recognizer, part of a recognition architecture, such as the depicted recognizer 46. Once trained, the recognizer 46 may be used to identify pathogens of interest to the system.

It will be understood that those of ordinary skill in the art of machine learning that the cell exposure, RNA extraction, microarray hybridization, training set development and training process may be part of an off-line process that is employed by the system 30 for building a recognizer 46. The recognizer 46 may be employed during on-line operations to identify a pathogen, such as the depicted pathogen 42 that can be applied to a set of host cells to infect those host cells and allow for RNA extraction 48. The off-line training and on-line operation of the system illustrated in FIG. 2 is depicted by the data flow diagram of FIG. 5. Specifically, FIG. 5 depicts a data flow diagram of one process for training and employing the recognizer. The extracted RNA may be applied to the microarray 50 and that test microarray 50 can, provide a pattern that can be processed by the recognizer 46 to generate an identification result 52 that is representative of the identity of the pathogen 42 as determined by the system 30.

Accordingly, FIG. 2 depicts that the system 30 can include an off-line learning process wherein one, or a plurality of pathogenic agents are exposed to host cells. As described above, the host cells may comprise a set of immortalized cells that provide a constant context for the reaction of the pathogen. The exposed host cells can be processed so that the resulting RNA can be extracted and applied to the microarrays 38A. The microarrays 38A can create a pattern related to the cell's response to the pathogenic agent, and that pattern can be used as a training point or vector within the microarray training set 40. In the embodiment depicted in FIG. 2, the microarray exposures related to cell RNA/cDNA. However, it will be understood by those of ordinary skill in the art, and it is described in more detail below, that the systems and methods described herein are not so limited. The systems and methods described herein can work with other types of data including proteomic data, metabolomic data and other kinds of data and combinations of such data. In any case, the data is provided as vectors to the microarray training set 40 which can include a plurality of training vectors related to a particular pathogenic agent, or, alternatively, a single vector related to the pathogenic agent. The number of training vectors developed for a pathogenic agent can vary according to the application and according to the available data for generating microarray training vectors.

The training process 42 depicted in FIG. 2 can be any suitable training process for creating the recognizer 46. Those of ordinary skill in the art of machine learning will know various techniques and practices that are suitable for generating the recognizer 46. Such techniques and practices are described in e.g., Fukunaga, K., Introduction to Statistical Pattern Recognition, Academic Press (1990) or Haykin, S., Neural Networks, Macmillan (1994), the contents of which are hereby incorporated by reference in their entirety.

Turning to FIG. 3, one particular embodiment is described for the purpose of illustrating certain embodiments of the invention. The system 60 in FIG. 3 is provided for illustration purposes only and is not to be understood as limiting in any way. FIG. 3 depicts a system 60 wherein training patterns 62 may be processed by an input space partitioning process 64. The input space partitioning process 64 can provide information for developing a training set 66 that recognizes differences in the effectiveness of certain subspaces within the input space, i.e., the multidimensional space where each dimension corresponds to the state (value) of a given microarray spot (probe). Thus, the input space partitioning process 64 divides available inputs (probe data) into subspaces. Each subspace may be characterized by a figure of fitness that represents the quality of the subspace from the discrimination viewpoint, i.e., predicts the quality of discrimination results provided by a classifier operating in that subspace. Techniques for partitioning the input space are described in Braun et al., Information Fusion of Large Numbers of Sources with Support Vector Machine Techniques, Proc. Of the SPIE, vol. 5099, April 2003, the contents of which are incorporated herein by reference in its entirety. Those of skill in the art will understand that the space partitioning processes described herein are merely examples of processes that can be used, and that other suitable processes maybe used without departing from the scope of the invention.

The recognizer 68 employed in FIG. 3 can include a plurality of subspace recognizers 70. Each of the subspace recognizers 70 may be associated with a respective one of the input space partitions. For each input partition, or subspace, the training process 66 trains the recognizer 68 so that a particular subspace recognizer 70 may be associated with a respective one of the input space partitions. For each input partition, or subspace, the training process 66 trains the recognizer 68 so that it has a particular subspace recognizer 70. That subspace recognizer 70 may be associated with a subspace measure of fitness that identifies or quantifies somewhat the general effectiveness of that subspace for recognizing and identifying pathogenic agents. FIG. 3 further depicts that in this particular embodiment, although not in all embodiments, the subspace recognizers 70 may include Support Vector Machine (SVM) recognizers. SVM methods are known in the art. References include J. J. Braun, Sensor Data Fusion with Support Vector Machine Techniques, Proc. of the SPIE, vol. 4731, April 2002, the contents of which are incorporated by reference.

In operation, the test pattern 74 may be applied to the recognizer 68, and each of the subspace recognizers 70 can generate a determination regarding the identity of the test pattern 74. Along with the recognizer results, the subspace recognizer 70 may report to the decision fuser 72 a recognizer measure of fitness. As discussed above, the recognizer measure of fitness can be a number that weighs or otherwise represents the effectiveness of the particular subspace recognizer or recognizing a pattern signature associated with a pathogenic agent.

The decision fuser 72 depicted in FIG. 3 can receive the recognizer results from each of the subspace recognizers 70 and the recognizer measure of fitness associated with each subspace recognizer and apply an information fusion technique for generating an identification of the test pattern. Accordingly, FIG. 3 depicts an example of a recognizer that has decision-level information fusion for allowing all or substantially all of the information provided by the test patterns and training patterns to be used in generating the identification of the pathogen. It will be noted by those of skill in the art that this type of information fusion is only one example of the information fusion that may be employed with the systems and methods described herein and in other embodiments, information fusion may be applied during the training process, or during any other process or combination of processes employed by the systems and methods of the invention.

In certain optional embodiments, the systems and methods described herein include dynamic measures of fitness. More specifically, in these optional embodiments, the knowledge of the training set enables generation of a measure of fitness that is dynamic in the sense of dependence on the specific test-points acquired at test-time, or in general a generation within each subspace of a “dynamic fitness measure” (DFM), which is a function of the training set and a given test point. In these embodiments, the recognition process involves classification and recognition-time DFM processing for each subspace, using multiple classifiers. Each subspace-level recognizer 70 shown in FIG. 3 operates on a projection of the test sample 74 (consisting of the probe data for a microarray that has been hybridized to by a material whose biological condition is to be determined) onto that subspace. The sample subspace-level classification results, the DFM values in each subspace for that sample, as well as the figures of fitness of the subspaces themselves are all forwarded to the final decision stage in fuser 72.

The final decision state fuses the above subspace outputs to form the discrimination result 22. The approaches to the final decision processing may vary and include a subspace-fitness-weighted fusion, a DFM-only based fusion, combined subspace-fitness and DFM-based fusion, and combined subspace-fitness and DFM-based fusion using a Dempster-Shafer Theory of Evidence. It should be noted that the final decision stage may include some or all of these methods and a selection of the method may be performed at recognition-time, based on the specifics of the recognition instance, such as whether all or only partial data is available.

The FIG. 4 depicts an alternate embodiment or practice of the invention wherein the system methods described herein are employed to perform a signature acquisition process wherein the signature of a pathogen is obtained and provided to the recognizer training process for adding the pathogen into the pathogen recognizer. Upon completion of the training process, the pathogen recognizer information can be uploaded to remote pathogen recognizers to allow for extending the recognition capability to remote recognizers. This allows the placement of any number of sensors in remote geographical locations. Moreover, it results in high extensibility, allowing easy addition of new pathogenic agents to the repertory of recognizable pathogenic agents, and electronic distribution of such capability and its extensions to multiple remote locations.

As described above, the systems and methods described herein include the combined: use of cultured cells as hosts (the resulting advantages including a high cell availability and reproducibility of their properties), the reliance on the in vitro expression patterns of these cell hosts during an in vitro encounter with pathogenic agents, microarray-based sensing, machine learning and microarray pattern recognition for automatic identification, and the use of information fusion methods (including methods for exploitation of all, or substantially all, available information and multiclassifier techniques, as well as the potential for using multiple host cell types and microarray types). Thus, one embodiment of this invention involves the combined use of all of the above components for automatic identification of pathogenic agents.

The methods described herein offer the following advantages:
High efficiency in that a single testing event (i.e., a single microarray exposure event) can check for multiple pathogens. In principle, this is only limited by how many pathogens the recognizer has been trained for. This is a substantial advantage over technologies that require individual tests for individual agents.
   The identification process does not require knowledge of the pathogen's genome.

The recognition apparatus training uses microarray patterns arising from host cells' exposure to the pathogen. As long as the host cell exhibits any response, the response patterns can be used for training and the knowledge of pathogen's genome is not required. This is beneficial as many existent pathogens have not yet been sequenced and newly emerging pathogens would also not have been sequenced when their identification becomes necessary. Furthermore, this method can be used to identify non-nucleic-acid-containing pathogens such as toxins and other chemical agents. As long as the host cell exhibits any response to the non-nucleic-acid-containing pathogenic agent, the response patterns can be used for training and neither the presence of genomic material nor knowledge of the pathogenic agent's chemical structure, etc., are required.
Any pathogen that elicits a non-stereotyped expression response in a host cell can in principle be identified with the described approach. The wide range of potentially identifiable pathogenic agents includes nucleic acid containing pathogenic agents, such as bacteria and viruses, as well as non-nucleic-acid-containing pathogenic agents such as toxins. This further differentiates this invention from approaches that rely directly on the pathogen's genome (e.g., PCR-based methods), since these approaches cannot be used to identify non-nucleic-acid-containing pathogenic agents or pathogens with an unknown genomic structure.
The identification process does not necessitate pathogen-specific materials, such as pathogen-specific microarrays. Rather, the approach exploits the high diversity of DNA probes within a typical microarray to provide, in aggregation, a distinguishable “signature” representing the host cell response.
It is expected that the approach should be robust to pathogen mutations and at least some malicious genetic engineering efforts. This stems from the use of host cells as the front-end of the identification process. Many mutations within the pathogenic genome will not alter the response pattern of the host cell. Many other mutations that do alter the response pattern of the host cells will not result in pattern alterations sufficiently large to affect the, statistical in nature, recognition process. Thus the host cell provides a filtering functionality, resulting in increased tolerance of the proposed method to pathogen mutations and engineering.
The repertory of recognizable pathogens is extendable simply by exposing the apparatus to samples containing a given pathogen. Automatic training of the recognition architecture results in the capability to recognize that pathogen.
The high extensibility of the approach goes beyond accommodating new pathogens. The product of the training process for new pathogen(s) is a computational entity, i.e., the data representing recognizer training results. These training results data sets can be distributed by electronic communication. Thus following the training for new pathogen (which may be performed at one location) recognizers worldwide can be upgraded to recognize the new pathogen without any physical or reagent changes, simply by means of electronic download.
The approach opens an avenue for handling unknown (uncataloged) as well as known (cataloged) pathogens. The following example scenario illustrates its power in case of unknown (uncataloged) pathogens. Consider a new disease outbreak at an arbitrary location A (the 2002 outbreak in the Far East that has later been termed SARS is a good analogy). In the initial phases of such an outbreak the causative agent is unknown, but systems and methods described herein could be used to acquire host cell patterns by exposing the apparatus to samples containing the pathogen (e.g., infected water, aerosol, or infected patients' body fluids) and collecting the host cell expression patterns. For this collection, the apparatus is neither in training nor recognition mode; it is merely acquiring the microarray patterns that include the new “pathogen X” signatures. Such patterns (obtained at a possibly distant location A) would be transmitted electronically to a development location D, where a recognizer retraining would be performed. Thus the capability to recognize “pathogen X” causative of the outbreak would be created. The retraining data could be transmitted to any recognizer worldwide, instantly upgrading it to be able to alert for “pathogen X”. Thus a suspicious event in any other location W could be tested for X. While this may or may not aid in eventual discovery of the nature of “pathogen X”, the ability to determine whether X is present at a given location W or the ability to determine conclusively that patient(s) at W (who may exhibit symptoms similar or different from those observed in location A) are or are not infected with X, is of value both clinically (treating those determined to be infected with X) and epidemiologically (public health aspects, prevention).

As a biodefense tool, this automatic and versatile multi-pathogen identification and classification method would be of interest to companies developing biodefense systems that require pathogen identification. For example, biodefense applications of the present invention include, but are not limited to, forensics and stand-alone bioattack detection systems. Moreover, the systems and methods described herein provide medical and/or veterinary diagnostics and public health tools and services, and provide a basis for versatile diagnostic procedure for identification of a wide range of pathogens from a wide variety of human or animal samples (e.g., sputum, smears, etc.) in clinical laboratory or hospital settings. Therefore, the invention will be of interest to biomedical and pharmaceutical industries involved in development of medical diagnostics products, including major pharmaceutical companies. The invention can provide a basis for diagnosing pathogen presence in a physician's office setting. The ability of the invention to track the presence of a pathogen responsible for an outbreak even when the nature of the pathogen is unknown is an additional advantage.

### APPLICATIONS

There are many potential applications of the methods and apparatuses of the present invention. For example, medical tests for various pathogenic infections performed individually (e.g., a separate test for each possible pathogen that may be responsible for the infection) are more time consuming, more expensive, and may require more sample from the patient. Additionally, when the physician or veterinarian must order each test individually, he must make a priori assumptions about the identity of the likely pathogen and then order particular tests based on those a priori assumptions. This approach increases costs while simultaneously increasing the risk of delayed or erroneous diagnoses. The present methods and apparatuses alleviate many of these limitations in the area of medical testing. By providing methods and apparatuses capable of simultaneously identifying any of a number of pathogenic agents, the present invention provides substantial improvements applicable to the field of medical testing. Pathogenic agents according to the present invention include both nucleic acid containing agents such as bacteria, viruses, fungi, and protozoa, as well as pathogenic agents that do not contain DNA, such as toxins. Exemplary nucleic acid containing agents include, but are not limited to, gram positive and gram negative bacteria, spore forming and non-spore forming bacteria, DNA-based viruses, and RNA-based viruses. Exemplary non-nucleic-acid-containing agents include, but are not limited to toxins and other toxic substances or stimuli.

A second application of the methods and apparatuses of the present invention is in the field of forensic science. Testing for presence of pathogenic agent, known or unknown (uncataloged), in a human, animal, or environmental sample, are examples of applications that can be addressed using the methods described herein.

Pathogenic agents according to the present invention include both nucleic acid containing agents such as bacteria, viruses, fungi, and protozoa, as well as non-nucleic-acid-containing agents. Exemplary nucleic acid containing agents include, but are not limited to, Gram positive and Gram negative bacteria, spore forming and non-spore-forming bacteria, DNA-based viruses, and RNA-based viruses. Exemplary non-nucleic acid containing agents include, but are not limited to, toxins. Samples suitable for analysis by the methods of the present invention include human or animal fluid or tissues including, but not limited to, blood, saliva, sputum, urine, feces, skin cells, hair follicles, semen, vaginal fluid, bone fragments, bone marrow, brain matter, cerebro-spinal fluid, amniotic fluid, and the like. Samples can be also obtained from various animals or plants. Samples may originate in any other substance, including but not limited to, soil, water, food, liquids, air or other gases, soot, fabrics, and the like.

By way of further example, the present invention can be used to screen blood, blood products, or other pre-packaged medical supplies to insure that these supplies are free from pathogenic agents such as bacteria, viruses, and toxin.

In addition to medical applications, the present invention has a variety of environmental uses. Samples may involve any substance, including but not limited to, soil, water, food, liquids, air or other gases, soot, fabrics, and the like. These samples can be analyzed for the presence of pathogenic agents. For example, samples of water collected from ponds, lakes, beaches, and reservoirs can be analyzed to assess the presence and concentration of bacteria, viruses, toxins, or pollutants. Such analysis can be used to monitor the health of these water sources and to evaluate their safety for human recreation and/or consumption. Similarly, samples of soil can be collected and analyzed to assess levels of contamination from natural or industrial sources.

Another application of the methods of the present invention is in the field of epidemiology. By facilitating the identification of pathogenic agents, including previously uncataloged pathogenic agents, the present invention provides substantial improvements in the epidemiology of disease and toxic contamination.

A final non-limiting example of applications of the present invention is in biodefense and homeland security, where the methods for pathogenic agent identification described herein can be used for detection or confirmation of biological attack, or for tracking the presence or spread of a biological agent. Features of the present invention that are particularly attractive for biodefense applications include, but are not limited to, the present methods' ability to operate with nucleic-acid containing pathogens as well as toxins, ability to operate with uncataloged pathogens, and the expected improved robustness to agent mutations or genetic engineering.

(ii) Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “sample” refers to a quantity of substance. Samples include, but are not limited to, substances that are gaseous, liquid, solid, organic, inorganic, biological, non-biological, environmental, industrial, and the like. Samples are obtained by any method of collection or preparation known in the art at the time of such collection or preparation. Exemplary samples include, but are not limited to, substances obtained from human, animal, or environmental sources or bodies. A sample can be derived from a human, plant, or animal. A sample can refer to particular soil, water, or other environmental sources. Exemplary samples also include, but are not limited to, humans; non-human animals including non-human primates, mice, rats, cats, dogs, cows, pigs, sheep, chickens, frogs, fish, goats, horses, and the like; plants including both dicots and monocots; environmental sources including soil, fresh water, salt water (e.g., streams, rivers, ponds, reservoirs, lakes, water bottles, the water supply of a building, etc), and air including an internal or external air supply (e.g., an air supply internal to a building, car, train, subway, plane, or other enclosed space as well as an external air supply).

Exemplary biological samples include, but are not limited to, blood, saliva, sputum, urine, feces, skin cells, hair follicles, semen, vaginal fluid, bone fragments, bone marrow, brain matter, cerebro-spinal fluid, and amniotic fluid. Exemplary environmental sample include, but are not limited to, soil, water, sewage, sludge, air, plant and other vegetative matter, oil, liquid mineral deposits, and solid mineral deposits.

The term “sample” also refers to any aliquot of any substance that possibly contains matter to be detected or identified by the present invention, in particular containing pathogenic agent.

Another use of the term “sample” entails a data item (number, point, or a multidimensional entity), representing an element of a set or collection of data items used in analytical, algorithmic or computational processes. Furthermore, the term “sample” is also used to describe a collection of data items (consisting of multiple, possibly multidimensional, entities) used in analytical, algorithmic or computational processes. Those trained in the art will readily comprehend a correct meaning of the term “sample,” according to one or more of the above instances, when the term “sample” is used in this invention.

The term “pathogenic agent” (herein used interchangeable with “pathogen”) refers to any potentially harmful agent capable of infecting a sample, or capable of eliciting a biological effect in host cells when such host cells are exposed to said pathogenic agent. Pathogenic agents according to the present invention include both nucleic acid containing agents such as bacteria, viruses, fungi, and protozoa, as well as non-nucleic-acid-containing substances, in particular toxins. Pathogenic agents also include agents whose impact on a sample or on host cells is mediated by either of a nucleic acid component and a non-nucleic-acid component (e.g., a bacteria that secretes a toxin). Exemplary nucleic acid containing agents include, but are not limited to, bacteria of any type, species or strain, viruses of any type, species or strain. Exemplary nucleic acid containing agents include, but are not limited to, Gram positive and Gram negative bacteria, spore-forming and non-spore-forming bacteria, DNA-based viruses, and RNA-based viruses. Exemplary non-nucleic-acid-containing agents include, but are not limited to, solid, gaseous, liquid, inorganic, organic, biological, or non-biological substances, or toxins.

The term “host cell” refers to any cell, capable of responding (e.g., capable of producing a biological response including, but not limited to, a gene expression response, proteomic response, a metabolic response, a morphological response, etc.) when said cell is exposed to a sample. The term “in vitro” refers to existence of the host cell outside any other organism, including but not limited to, existence as a single cell or a set of cells within an enclosure or chamber. The term “exogenous” is also used to described such organism-independent existence of the host cells. The response, used interchangeably with “in vitro response” and “in vitro expression response”, refers to any reaction of the host cell to the presence of a sample when such sample contacts or otherwise interacts with the host cell. In particular, in this invention the host cell response is used to measure the physiological impact of a pathogenic agent. The term “cultured host cell” refers to the host cells that can be grown in vitro, in order to create multiplicity of host cells. This includes, but is not limited to cells originating from cell lines. Any cell can be used as a host cell, and the invention includes the use of test cells derived from any of a number of organisms and/or tissue types. The host cell may be derived from the same species as the source from which the sample is taken, however, the host cell may also be derived from a different species. All that is necessary is that a sample containing a pathogenic agent elicits a change in some measurable biological parameter in a host cell (e.g., a change in gene expression, protein expression, cell shape, cell conductivity, cell metabolic activity, etc.).

Exemplary host cells include commercially available transformed cell lines, commercially available non-transformed cell lines, and primary cell cultures. Exemplary host cells include, but are not limited to, cells derived from humans, non-human animals, plants, bacteria, yeast, etc. Furthermore, exemplary host cells derived from human or non-human animals include cells derived from any of a number of tissues including, but not limited to, neuronal tissue, bone marrow, skin and other epithelial tissue, endothelial tissue, lung, myocardium, pericardium, endocardium, blood, immune system cells, liver, pancreatic tissue, kidney, gall bladder, stomach, small intestine, large intestine, bladder, esophageal tissue, tongue, retina, and the like. Furthermore, exemplary host cells may be derived from healthy tissue or diseased tissue (e.g., cancerous tissue, etc). We note that methods of growing and maintaining a wide range of primary and immortalized cells in culture are well known in the art, and one of skill in the art can perform these tasks using techniques available in the art.

Host cells of the present invention are used to “amplify” and “filter” the effect of a pathogenic agent. Contacting test cells with a sample containing a pathogenic agent “amplifies” in the sense that a small quantity of a virulent pathogen may result in a vigorous expression response in the host cells.

Host cells also serve as a “filter” of differences in the pathogen's genome, such as those caused by some mutations or genetic engineering, since the host expression response pattern is likely to be similar if these differences do not significantly alter the pathogen-host interaction.

Standard methods of pathogen identification rely on detection of a nucleic sequence of the pathogen itself. Methods that rely on detection of a pathogen nucleic acid sequence are vulnerable to pathogen mutation since even single nucleic acid mutations may prevent detection. The term “filter” or “filtering” is specifically relevant to this issue and refers to the relative resilience of the present identification methods to pathogenic mutation. The present methods rely on detection of changes in gene or protein expression in an affected host cell. These changes in gene or protein expression in the host cell are indicative of the pathogenic agent, and thus would not be expected to change dramatically in response to small variations or mutations in pathogenic nucleic acid sequence. Accordingly, the host cells used to measure the cellular effect of the pathogenic agent (i) serve as a filter for small variations or polymorphisms across a population of pathogenic agents, (ii) serve as a filter for mutation in a pathogenic agent, and (iii) serve as a filter of some genetic engineering effects.

“Amino acid”—a monomeric unit of a peptide, polypeptide, or protein. There are twenty amino acids found in naturally occurring peptides, polypeptides and proteins, all of which are L-isomers. The term also includes analogs of the amino acids and D-isomers of the protein amino acids and their analogs.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

As used herein, the term “nucleic acid” refers to polynucleotides such as deoxyribonucleic acid (DNA), and, where appropriate, ribonucleic acid (RNA). The term should be understood to include single (sense or antisense) and double-stranded polynucleotides.

As used herein, the term “hybridization” refers to a phenomenon that involves a binding event between entities or molecules exposed to a microarray probe (spot) to entities or molecules within the probe (spot) of the microarray, including such binding occurring simultaneously in multiple probes (spots) of the microarray. A particular instance of hybridization involves labeled cDNA to oligomer probes (spots) of a DNA microarray (also referred to as genomic microarray, gene chip, and the like). Other modalities exist, including, but not limited to, proteomic microarrays. Techniques of microarray processing, including hybridization, are known in the art.

Those of skill in the art will understand that the depicted recognizer 46 can run on a data processing system that can be a conventional data processing platform such as an IBM PC-compatible computer running the Windows operating systems, or a SUN workstation running a Unix operating system. Alternatively, the data processing system can comprise a dedicated processing system that includes an embedded programmable data processing system that can include the pathogen identification mechanism. For example, the data processing system can comprise a single board computer system that has been integrated into a system for performing pathogen identification. The single board computer (SBC) system can be any suitable SBC, which include microprocessors, data memory and program memory, as well as expandable bus configurations and an on-board operating system.

In a further alternative embodiment, the data processing system can comprise a micro-controller system that can comprise the identification system 30. The micro controller system can also be embedded into an assay processing system, such as the above-mentioned system. The micro-controller can comprise a commercially available micro-controllers. The micro controllers can execute programs for implementing the image processing, information fusion, and pattern recognition functions as well as for controlling the elements of the assay system, such as by executing motor control processes. Optionally, the data processing system can also include signal processing systems for performing the image processing. These systems can include any of the digital signal processors (DSP) capable of implementing the image processing functions described herein, such as the DIPS based on the TMS320 core including those sold and manufactured by the Texas Instruments Company of Austin, Tex.

Accordingly, although FIGS. 1 and 3 graphically depict the identification mechanisms 10 and 60 as comprising functional block elements, it will be apparent to one of ordinary skill in the art that these elements can be realized as computer programs or portions of computer programs that are capable of running on the data processor platform to thereby configure the data processor as a system according to the invention. Moreover, although FIG. 1 depicts the system 10 as an integrated unit, it will be apparent to those or ordinary skill in the art that this is only one embodiment. Accordingly, it is not necessary that the identification system be directly coupled to a data processing system, and instead the data generated by the microarrays 14 can be imported into a data processing system by any suitable technique, including by file transfer over a computer network, or by storing the microarray data file on a disk and mounting copying the disk into the file system of the data processing system 12. Thus it will be apparent that the microarray system can be remote from the recognizer. Furthermore, the training process of the identification architecture can be separate from the identification (recognition) process. Thus, the invention includes embodiments, wherein users at multiple remote sites create data and deliver the data to a remote processing system that can identify and interpret the patterns, or training performed in one or more locations can be used to extend the identification of any number of remote identification systems to which the training results can be distributed by electronic communication means.

As discussed above, the pathogen identification system can be realized as a software component operating on a conventional data processing system such as a PC or Unix workstation. In that embodiment, the system can be implemented as a computer program written in any language including, but not limited to, Matlab, C, C++, Java, or Fortran. Additionally, in an embodiment where microcontrollers or DSPs are employed, the system can be realized as a computer program written in microcode or written in a high-level language and compiled down to microcode that can be executed on the platform employed. The development of such systems is known to those of skill in the art Other hardware architectures and their corresponding operating system infrastructures, including, but not limited to, parallel computer architectures, are noted as applicable platforms for the present invention.

Those skilled in the art will know or be able to ascertain using no more than routine experimentation, many equivalents to the embodiments and practices described herein. Accordingly, it will be understood that the invention is not to be limited to the embodiments disclosed herein, but is to be understood from the following claims, which are to be interpreted as broadly as allowed under the law. All publications, patents and patent applications are herein incorporated by reference in their entirety to the same extent as if each individual publication, patent or patent application was specifically and individually indicated to be incorporated by reference in its entirety.

1. A method of constructing an apparatus for identifying a pathogenic agent in a sample, comprising
providing a set of host cells and contacting the cells with the pathogenic agent or any sample containing pathogenic agent,
employing a microarray having a plurality of probes to measure a plurality of biological responses of the host cells,
applying the measured plural biological responses to train a machine learning system to recognize a pathogenic agent, and
detecting and identifying a pathogenic agent in a sample, by exposing host cells to said sample, using a microarray to measure plural biological responses provoked in host cells, and employing the trained machine learning system to identify the pathogenic agent.

2. A method according to claim 1, further comprising
employing the set of host cells and a plurality of microarrays to increase a plurality of biological responses available to the identification process, and
applying machine learning to said plural biological responses to identify a pathogenic signature.

3. A method according to claim 2, further comprising
providing a plurality of sets of host cells,
contacting said host cells with a sample containing pathogenic agents to provoke and measure a plurality of biological responses,
training a recognizer to detect one or more of said pathogenic signatures in a biological response provoked in a host cell, and
applying machine learning to said plural respective biological responses to identify at least one pathogenic signature.

4. A method according to claim 1, further comprising
wherein the pathogenic agent to be identified is contained within an environmental sample that contains other substances and/or pathogenic agents.

5. A method according to claim 1, further comprising
employing substantially all of the measured biological response data during the identification method to widen the scope of information employed during pathogen detection.

6-9. (canceled)

10. A method according to claim 1, further comprising
employing the similarity of the host cell response to pathogenic agents that represent different strains of the same pathogen, altered pathogens, genetically engineered pathogens, and/or mutated pathogens,
wherein the host cells act as a natural filtering mechanism allowing identification of the pathogenic agents that differ from the agents used for training

11. A method according to claim 1, wherein
employing a microarray includes employing a microarray having a uniform set of probes.

12-13. (canceled)

14. A method according to claim 1, wherein the host cells include cultured host cells and/or host cells grown from cell lines.

15. A method according to claim 1, wherein the host cells include host cells of different types

16. A method according to claim 1, wherein host cells include cells selected from different organisms or species.

17-18. (canceled)

19. The method according to claim 1, wherein the pathogenic agent includes substances and/or stimuli capable of eliciting a response in the host cell.

20. The method of claim 1, wherein the sample is derived from a human or animal, and wherein the sample is selected from the group consisting of blood, urine, feces, sputum, saliva, semen, vaginal fluid, cerebrospinal fluid, skin cells, hair follicles, bone fragments, bone marrow, brain matter, and amniotic fluid.

21. The method of claim 1, wherein the sample is derived from an environmental or industrial matter.

22. The method of claim 1, wherein the sample consists of gas, liquid, or solid, or combinations of these states.

23. The method of claim 1, wherein the sample is selected from the group of air, water, and soil.

24. The method of claim 1, wherein the host cells comprise a cell selected from the group of lung, skin, nerve, and immune system.

25. The method of claim 1, wherein the one or more biological responses of the host cells comprise genomic microarray data of the host cell response.

26. The method of claim 1, wherein the one or more biological responses of the host cells comprise proteomic microarray data of the host cell response.

27. The method of claim 1, wherein the one or more biological responses of the host cells comprise both genomic microarray data and proteomic microarray data

28. The method of claim 1, wherein the one or biological responses of the host cells comprise genomic, proteomic, metabolomic and fusion thereof.

29. The method of claim 1, wherein the microarrays include microarrays having non-uniform probe sets, or multiple microarrays having different sets of probes.

3034. (canceled)

35. The method of claim 7,
wherein fusing includes weighting candidate identification responses.

36. A method for identifying the presence of a pathogenic agent, comprising
collecting disparate types of biological data representative of a biological response to the same pathogenic agent, and
employing information fusion to process the biological response.

37. The method of claim 36, including the further step of
collecting multiple modalities of biological data representative of a biological response to the same pathogenic agent.

38. The method of claim 36, wherein collecting data includes
employing microarrays having a set of probes.

39. The method of claim 36, further comprising
applying machine learning to process the biological data and to develop a signature for the pathogen that includes substantially all of the data collected by common probes among the microarrays.

40. The method of claim 36, wherein
the biological response include the biological response of a host cell.

41-46. (canceled)

47. The method of claim 36, wherein collecting disparate types of biological data representative of a biological response comprises
providing a set of host cells and contacting the cells with the pathogenic agent or any sample containing pathogenic agent and
employing a microarray having a plurality of probes to measure a plurality of biological responses of the host cells, and wherein the method further includes
applying the measured plural biological responses to train a machine learning system to recognize the pathogenic agent and
detecting and identifying the pathogenic agent in a sample, by exposing host cells to said sample, using a microarray to measure plural biological responses provoked in host cells, and employing the trained machine learning system to identify the pathogenic agent.

48. The method of claim 47, further comprising
employing the set of host cells and a plurality of microarrays to increase a plurality of biological responses available to the identification process, and
applying machine learning to said plural biological responses to identify a pathogenic signature.

49. The method of claim 48, further comprising
providing a plurality of sets of host cells,
contacting said host cells with a sample containing pathogenic agents to provoke and measure a plurality of biological responses,
training a recognizer to detect one or more of said pathogenic signatures in a biological response provoked in a host cell, and
applying machine learning to said plural respective biological responses to identify at least one pathogenic signature.

50. The method of claim 47, further comprising
employing substantially all of the measured biological response data during the identification method to widen the scope of information employed during pathogen detection.

51. The method of claim 47, wherein
employing substantially all of the measured biological response data includes identifying a pathogen signature having substantially all of the measured biological data.

52. The method of claim 49, further comprising
allowing the recognizer to generate plural decision results, and
fusing said plural decision results to generate a determination of the identity of a pathogen in a test sample.

53. The method of claim 47, further comprising
using the host cells as a natural amplification mechanism, wherein the host cell response to an agent of high virulence is vigorous, allowing improved detection and identification of pathogenic agents.

54. The method of claim 47, wherein
employing a microarray includes employing microarrays of different modalities.

55. The method of claim 54, wherein the different modalities include modalities selected from the group consisting of genomic, proteomic, and metabolomic.

56. The method of claim 47, wherein the pathogenic agent is a non-nucleic-acid-containing pathogenic agent.

57. The method of claim 56, wherein the non-nucleic-acid-containing pathogenic agent is a toxin.

58. The method of claim 47, further comprising
fusing information from multiple types and/or species of host cells, multiple microarray types, multiple and/or disparate sets of probes, and/or multiple modalities.

59. The method of claim 47, further comprising
fusing multiple candidate identification responses generated by multiple classifiers.

60. The method of claim 47, further comprising
partitioning an input space of microarray probes into one or more computational subspaces and generating measures of fitness for said subspaces.

61. The method of claim 47, further comprising
generating multiple measures of fitness within a subspace wherein intra-subspace measures of fitness are dynamic having a value depending on the region within the subspace and position within the subspace of a point representing the test sample

62. The method of claim 60, further comprising determining for a subspace a fitness measure representative of an effectiveness of a classifier operating in the respective subspace.

63. The method of claim 47, wherein analyzing a data set includes partitioning an input space into plurality of subspaces.

64. The method of claim 63, further comprising fusing measures of recognition generated from respective areas of said subspaces.

65. The method of claim 64, further comprising using subspace measures of fitness and fusing multiple classifiers.

66. The method of claim 65, further comprising
applying Dempster-Shafer theory of evidence for fusing multiple classifiers.
